(19) United States
(12) Patent Application Publication
(10) Pub. No.: US 2024/0096463 A1
(43) Pub. Date: Mar. 21, 2024

(54) TECHNIQUES FOR USING A HYBRID MODEL FOR GENERATING TAGS AND INSIGHTS

(71) Applicant: Oura Health Oy, Oulu (FI)

(72) Inventor: Jukka Partanen, Mäntsälä (FI)

(21) Appl. No.: 17/946,943

(22) Filed: Sep. 16, 2022

(51) Int. Cl. G16H 10/65; G06F 16/29; G16H 40/63

(52) U.S. Cl. CPC G16H 10/65; G06F 16/29; G16H 40/63

(57) ABSTRACT

Methods, systems, and devices for taggable event detection are described. A system may receive geographical location data associated with a user throughout a time interval, and receive physiological data associated with the user from a wearable device. The system may correlate the physiological data with candidate taggable events, where the candidate taggable events are associated with respective confidence values that indicate confidence levels that the corresponding candidate taggable events occurred within the time interval. The system may selectively modify the confidence values associated with the candidate taggable events based on the geographical location data to generate one or more modified confidence values, and identify a taggable event within the time interval based on a modified confidence value associated with the taggable event satisfying a threshold confidence value. The system may then cause a graphical user interface (GUI) of a user device to display an indication of the identified taggable event.

### FIELD OF TECHNOLOGY

The following relates to wearable devices and data processing, including techniques for using a hybrid model to generate tags and insights.

### BACKGROUND

Some wearable devices may be configured to collect physiological data from users. Acquired physiological data may be used to provide health-related insights to the user. However, conventional techniques for providing health-related insights may not accurately identify relationships between the user's activities and their physiological data, and may therefore be deficient.

### BRIEF DESCRIPTION OF THE DRAWINGS

FIG. 1 illustrates an example of a system that supports techniques for using a hybrid model for generating tags and insights in accordance with aspects of the present disclosure.

FIG. 2 illustrates an example of a system that supports techniques for using a hybrid model for generating tags and insights in accordance with aspects of the present disclosure.

FIG. 3 illustrates an example of a system that supports techniques for using a hybrid model for generating tags and insights in accordance with aspects of the present disclosure.

FIG. 4 illustrates an example of a timeline that supports techniques for using a hybrid model for generating tags and insights in accordance with aspects of the present disclosure.

FIG. 5 illustrates an example of a graphical user interface (GUI) that supports techniques for using a hybrid model for generating tags and insights in accordance with aspects of the present disclosure.

FIG. 6 shows a block diagram of an apparatus that supports techniques for using a hybrid model for generating tags and insights in accordance with aspects of the present disclosure.

FIG. 7 shows a block diagram of a wearable application that supports techniques for using a hybrid model for generating tags and insights in accordance with aspects of the present disclosure.

FIG. 8 shows a diagram of a system including a device that supports techniques for using a hybrid model for generating tags and insights in accordance with aspects of the present disclosure.

FIGS. 9 through 12 show flowcharts illustrating methods that support techniques for using a hybrid model for generating tags and insights in accordance with aspects of the present disclosure.

### DETAILED DESCRIPTION

Wearable devices may be used to collect, monitor, and track physiological data associated with a user based on sensor measurements performed by the wearable device. Examples of physiological data may include temperature data, heart rate data, photoplethysmography (PPG) data, and the like. The physiological data collected, monitored, and tracked via the wearable ring device may be used to gain health insights about the user, such as the user's sleeping history, activity history, physiological history, and the like. However, existing techniques for providing insights to a user based on their sleeping history, activity history, physiological history, or their general preferences may fail to identify and provide valuable insights that enable users to make changes in their lives to improve their overall physical health. For example, although wearable devices may be able to determine that a user is suffering from poor sleep quality, such wearable devices may be unable to determine the underlying factors or user behaviors that are causing the poor sleep quality. In other words, wearable devices may be unable to draw relationships between user behaviors and resulting physiological responses.

Some wearable devices may enable users to manually input “tags” or other otherwise input indications of certain events, activities, or conditions via a mobile device. Taggable events may include, but are not limited to, alcohol consumption, caffeine consumption, travel, a workout, late night meal, etc.). Inputting such tags may enable a system to more accurately and efficiently gain insights regarding how a user's behavior and actions affect their physiological data. For example, if a user tags caffeine consumption in the late afternoon, and subsequently suffers from poor sleep quality that night, the system may be able to conclude that the user's poor sleep quality is due to late caffeine consumption, and may therefore suggest that the user reduce their caffeine consumption or adjust a timing of their caffeine consumption in order to improve their sleep quality.

However, in some cases, it may be inconvenient or impossible for a user to access a mobile device to input tags and other information, such as in cases where the mobile device is located some distance from the user. The inability to access the mobile device (e.g., an application executable on the mobile device) may prevent the user from inputting such tags. Moreover, even in cases where users are able to easily access a mobile device, requiring users to open up a wearable application to manually input tags associated with a wearable device may significantly reduce a frequency that users input information. Furthermore, conventional wearable devices have been unable to efficiently and reliably tag certain events, activities, or conditions without manual input from the user.

Accordingly, aspects of the present disclosure are directed to a hybrid model for generating tags and insights related to a user. In particular, techniques described herein may utilize physiological data collected from a wearable device, as well as other data types/sources, to more efficiently and reliably identify taggable events (e.g., caffeine consumption, alcohol consumption, etc.) that may be used to determine health-related insights for a user. Other types and sources of data that may be used to improve identification of taggable events may include, but are not limited to, geographical location data (e.g., where taggable events are likely to occur), time of day data (e.g., when taggable events are likely to occur), and the like.

For example, in some cases, geographical location data associated with a user may be leveraged to identify taggable events for a user. In this example, a system (e.g., wearable device, user device, etc.) may acquire geographical location data for a user over a time interval, such as through a global positioning system (GPS), via a calendar application executable on the user device, and the like. Additionally, a wearable device (e.g., wearable ring device) may collect physiological data associated with a user. The system may correlate the acquired physiological data (e.g., heart rate data, motion data, temperature data, PPG data, etc.) with one or more candidate taggable events. For example, in cases where the wearable device collects high temperature and high heart rate readings from the user, the system may correlate the acquired physiological data with a workout or other exercise as well as caffeine and/or alcohol consumption, in which case the workout/exercise, caffeine consumption, and alcohol consumption are each considered to be candidate taggable events. Each candidate taggable event may be associated with a relative confidence value indicating a confidence level that the respective taggable event occurred and is correlated with the acquired (e.g., a relative probability or likelihood that the taggable event occurred).

Continuing with the same example, the system may selectively modify the confidence values associated with the candidate taggable events based on the geographical location data. For example, in cases where the system identifies that the user was at a bar before or during the acquired physiological data that indicated high temperature and heart rate readings, the system may determine that it is more likely that user consumed alcohol that resulted in the high temperature/heart rate readings, and that it is less likely that the user performed a workout or consumed caffeine while at the bar. Accordingly, the system may selectively increase the confidence value associated with alcohol consumption, and selectively decrease the confidence values associated with caffeine consumption and exercise. In this example, the system may determine that the modified confidence value for alcohol consumption satisfies some threshold, and may therefore identify and tag “alcohol consumption” as a taggable event for the user. Subsequently, the identified taggable event (e.g., alcohol consumption) may be used to further improve and provide health-related insights to the user (e.g., determine how alcohol consumption affects the user's physiological data, and provide recommendations to the user accordingly).

Additionally, or alternatively, data associated with the relative time of day (e.g., early morning, mid-day, afternoon, evening, late night) may be leveraged to reliably identify taggable events for a user. Such time of day data may be used in addition to, or in the alternate to, the geographical location data described above. For instance, similar to the example above, a wearable device may collect physiological data for a user during the early morning (e.g., 6 am to 7 am), where the collected physiological data includes high temperature and high heart rate readings for the user. The system may correlate the acquired physiological data with a workout/exercise, as well as caffeine and/or alcohol consumption (e.g., identify a workout, caffeine consumption, and alcohol consumption as candidate taggable events). In this example, the system may selectively modify the confidence values associated with the candidate taggable events based on the time of day that the physiological data was acquired (e.g., early morning). In this regard, the system may determine that it is more likely that the user consumed caffeine in the early morning that resulted in the high temperature/heart rate readings, and that it is less likely that the user performed a workout or consumed alcohol so early in the morning. Accordingly, the system may selectively increase the confidence value associated with caffeine consumption, and selectively decrease the confidence values associated with alcohol consumption and exercise. As such, the system may determine that the modified confidence value for caffeine consumption satisfies some threshold, and may therefore identify and tag “caffeine consumption” as a taggable event for the user, where the taggable event may be used to further improve and provide health-related insights to the user.

Aspects of the disclosure are initially described in the context of systems supporting physiological data collection from users via wearable devices. Aspects of the disclosure are additionally described in the context of an example timeline and an example GUI. Aspects of the disclosure are further illustrated by and described with reference to apparatus diagrams, system diagrams, and flowcharts that relate to techniques for using a hybrid model for generating tags and insights.

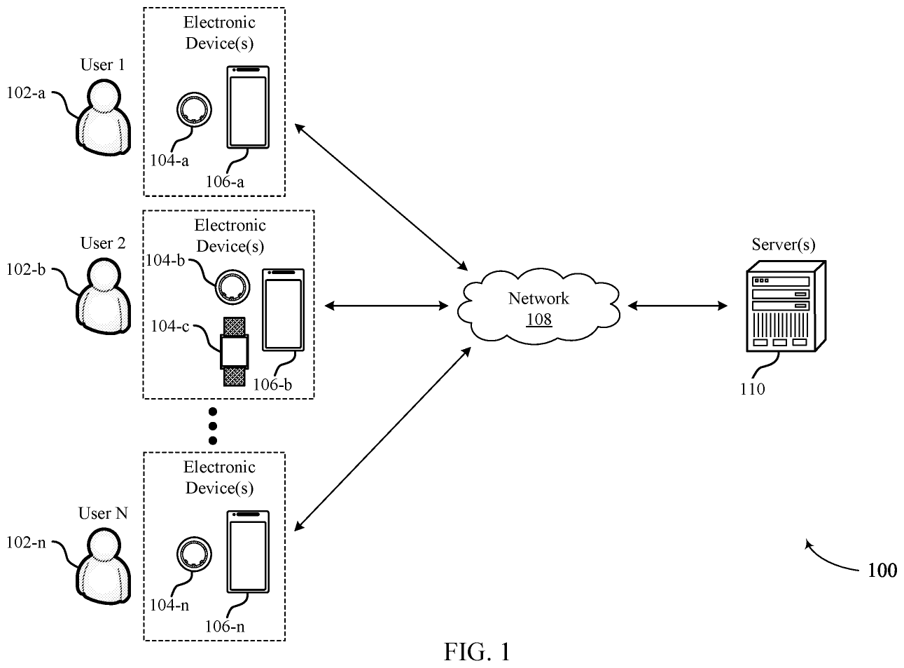

FIG. 1 illustrates an example of a system 100 that supports techniques for using a hybrid model for generating tags and insights in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for implementing a hybrid model for generating tags and insights related to a user 102. In particular, techniques described herein may utilize physiological data collected from a wearable device 104, as well as other data types/sources, to more efficiently and reliably identify taggable events (e.g., caffeine consumption, alcohol consumption, etc.) that may be used to determine health-related insights for a user 102. Other types and sources of data that may be used to improve identification of taggable events may include, but are not limited to, geographical location data (e.g., where taggable events are likely to occur), time of day data (e.g., when taggable events are likely to occur), and the like.

Stated differently, the components of the system 100 may be configured to leverage other data sources to determine a relative likelihood or probability that the user engaged in certain activities (e.g., relative probability of taggable events). By more accurately and reliability identifying taggable events for the user 102, the system 100 may be able to draw health-related insights regarding how the user's behaviors and tendencies (e.g., taggable events) affect the user's overall health.

For example, the system 100 may be configured to correlate physiological data collected from a user 102 via a wearable device 104 with candidate taggable events, such as a workout, caffeine consumption, alcohol consumption, etc., where each respective candidate taggable event is associated with a corresponding confidence value. According to techniques herein, the system 100 may be configured to selectively modify the confidence values of the respective candidate taggable events according to other types/sources of data, such as geographical location data, time of day data, and the like. In other words, the system 100 may be configured to evaluate when and where candidate taggable events are likely to occur, and adjust confidence values of the candidate taggable events accordingly. For instance, the system 100 may be configured to determine that it is likely the user consumed alcohol while at a bar. By way of another example, if a user typically exercises on their lunch break, the system 100 may be configured to determine that the user likely went on a run due to their elevated heart rate over the lunch hour.

By leveraging other data sources to automatically identify taggable events for a user 102, techniques described herein may result in more frequent and reliable identification of taggable events. Moreover, by improving the ability of the system 100 to identify taggable events, techniques described herein may enable the system 100 to identify relationships between taggable events and the user's physiological data (e.g., late workouts for the first user 102-a result in poor sleep quality, etc.). Such relationships may be used to generate health-related insights and recommendations that may be provided to the users 102 (e.g., via a graphical user interface (GUI) of a user device 106) to enable the users 102 to adjust their habits and tendencies in order to improve their overall health.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

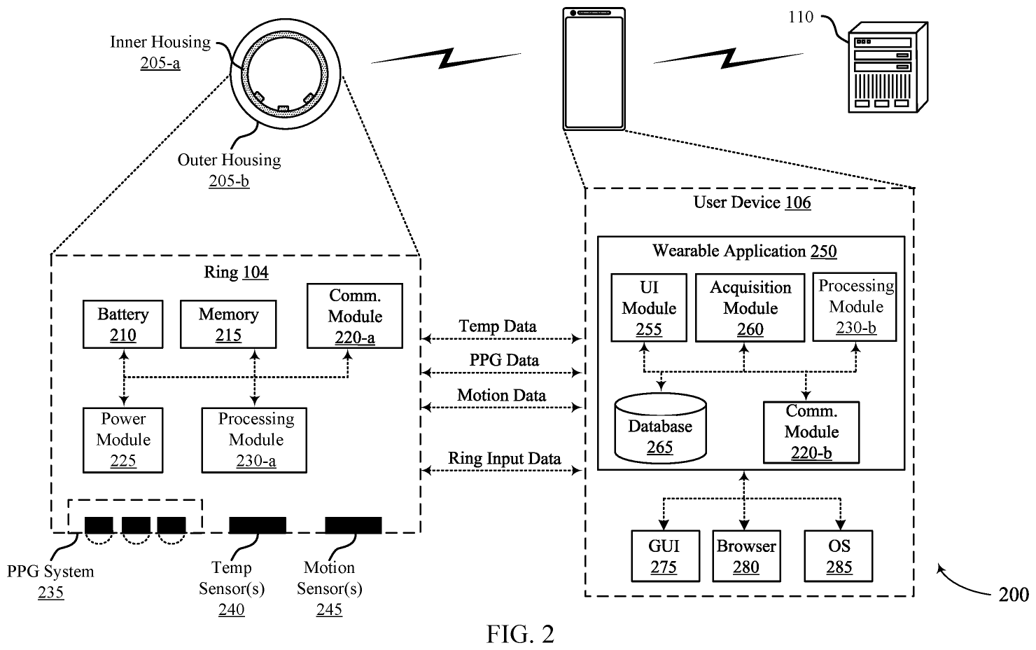

FIG. 2 illustrates an example of a system 200 that supports techniques for using a hybrid model for generating tags and insights in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques for implementing a hybrid model for generating tags and insights related to a user 102. In particular, techniques described herein may utilize physiological data collected from a wearable device 104, as well as other data types/sources, to more efficiently and reliably identify taggable events (e.g., caffeine consumption, alcohol consumption, etc.) that may be used to determine health-related insights for a user 102. Other types and sources of data that may be used to improve identification of taggable events may include, but are not limited to, geographical location data (e.g., where taggable events are likely to occur), time of day data (e.g., when taggable events are likely to occur), and the like.

In other words, the system 100 may be configured to leverage other data sources to determine a relative likelihood or probability that the user engaged in certain activities (e.g., relative probability of taggable events). Other types and sources of data that may be used to improve identification of taggable events may include, but are not limited to, geographical location data (e.g., where taggable events are likely to occur), time of day data (e.g., when taggable events are likely to occur), and the like. By more accurately and reliably identifying taggable events for the user 102, the system 100 may be able to draw health-related insights regarding how the user's behaviors and tendencies (e.g., taggable events) affect the user's overall health.

For example, the wearable device 104, the user device 106, the servers 110, or any combination thereof, may collect geographical location data for a user (e.g., determine where the user is located). For example, the user device 106 may receive the geographical location data via a GPS associated with the system 200. By way of another example, the user device 106 may access calendar events included within a calendar application executable on the user device 106 to determine geographical location for the user (e.g., a calendar event indicating “tennis” may be used to determine that the user is likely located at a tennis club or fitness center for the duration of the calendar event). As such, the user device 106 may identify a location of the user based on the geographical location data and a time interval that the user is (or was) located at the location.

Continuing with the same example, the wearable device 104 may acquire physiological data from the user. In some aspects, the wearable device 104, the user device 106, the servers 110, or any combination thereof, may correlate the collected physiological data with candidate taggable events, such as a workout, caffeine consumption, alcohol consumption, etc. Moreover, one or more components of the system 100 may calculate a confidence value associated with each respective candidate taggable event, where the confidence values indicate a relative likelihood or probability that the candidate taggable event occurred. The confidence values may be based on any number of factors including, but not limited to, a relative quality of the acquired physiological data, historical physiological data associated with the user, historical taggable events identified for the user, and the like. In some implementations, a classifier or other machine learning algorithm may be used to identify the candidate taggable events, determine the confidence values, and the like. Additionally, or alternatively, the confidence values may be based on previous tags or user inputs received from the user (e.g., historical tags or historical taggable event data). For example, the confidence values may based on previous tags/taggable events indicating that the user has previously performed a taggable event in a specific location, in a specific physiological situation (e.g., exercise), and the like.

In this example, one or more components of the system 200 may be configured to selectively modify the confidence values of the respective candidate taggable events according to other types/sources of data. Other types/sources of data that may be used to selectively adjust the confidence values may include, but are not limited to, geographical location data (e.g., where taggable events are likely/expected to occur), time of day data (e.g., when taggable events are likely/expected to occur), and the like. In other words, the system 100 may be configured to evaluate when and where candidate taggable events are likely to occur, and adjust confidence values of the candidate taggable events accordingly. For instance, the system 100 may be configured to determine that it is likely the user consumed alcohol while at a bar. By way of another example, if a user typically exercises on their lunch break, the system 100 may be configured to determine that the user likely went on a run due to their elevated heart rate over the lunch hour.

Subsequently, the system 100 (e.g., wearable device 104, user device 106, servers 110) may identify a taggable event based on a modified confidence value corresponding to a candidate taggable event satisfying some threshold. In some implementations, a machine learning model (e.g., classifier) may be used to modify confidence values and identify taggable events. Upon identifying a taggable event, the GUI 275 of the user device 106 may display an indication of the identified taggable event to the user. In some aspects, the user may be able to confirm or deny the taggable event, modify the taggable event, provide supplemental information associated with the taggable event, or any combination thereof.

By leveraging other data sources to automatically identify taggable events for a user 102, techniques described herein may result in more frequent and reliable identification of taggable events. Moreover, by improving the ability of the system 100 to identify taggable events, techniques described herein may enable the system 100 to identify relationships between taggable events and the user's physiological data (e.g., late workouts for the first user 102-a result in poor sleep quality, etc.). Such relationships may be used to generate health-related insights and recommendations that may be provided to the users 102 (e.g., via a graphical user interface (GUI) of a user device 106) to enable the users 102 to adjust their habits and tendencies in order to improve their overall health.

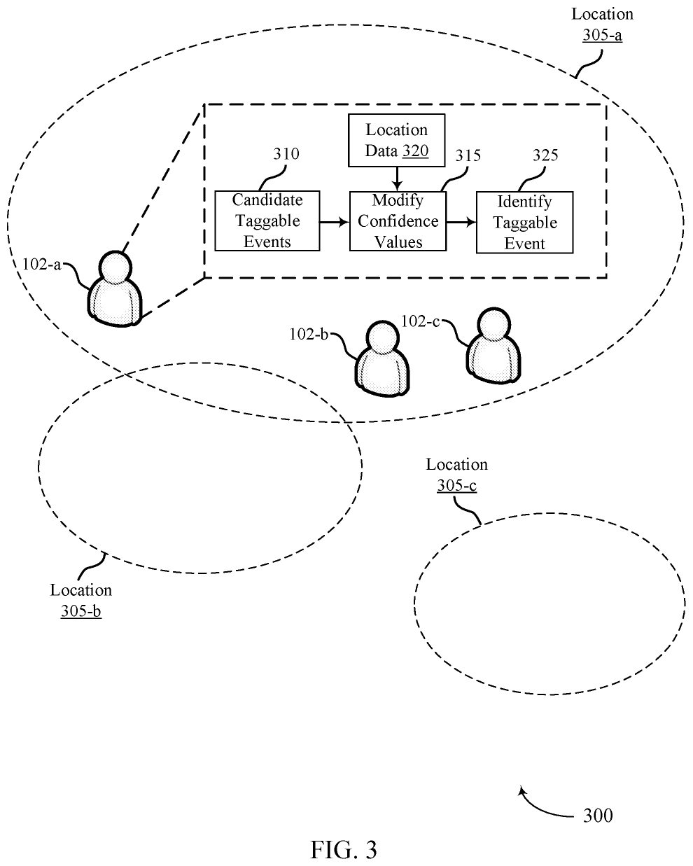

FIG. 3 illustrates an example of a system 300 that supports techniques for using a hybrid model for generating tags and insights in accordance with aspects of the present disclosure. The system 300 may implement, or be implemented by, aspects of the system 100, the system 200, or both.

The system 300 shown in FIG. 3 illustrates an example of multiple locations 305, including a location 305-a, a location 305-b, and a location 305-c that a user 102 may be located (e.g., such as a bar, a swimming pool, a restaurant, a gym, or the like thereof). In some cases, the location 305 may be associated with a zone or other geographical boundary, as depicted by the dotted lines, such that the user 102 may be considered “at the location 305” when located within the zone. Additionally, some locations 305, such as the location 305-a and the location 305-b, may overlap (e.g., the respective zones may overlap) such that the user 102 may be considered to be at both or either of the location 305-a and the location 305-b when located in the overlapping zone.

In some aspects, the system 300 may acquire geographical location data 320 associated with a location 305 of the user 102 throughout a time interval. That is, the system 300 may receive an indication of the location 305 (e.g., semantic location 305) that the user 102 has been located for a time threshold (e.g., the time interval). Geographical location data 320 associated with the user may be collected via a wearable device 104 associated with the user, a user device 106 (e.g., smartphone, smart watch, etc.) In some cases, the geographical location data may include GPS data acquired via a user device 106. Additionally, or alternatively, the system 200 may determine a location of the user (e.g., acquire geographical location data 320) via a calendar application executable by the user device 106, or may be obtained by any other means that the location 305 of the user 102 may be determined.

For example, a calendar application executable by a user device 106 may indicate an event (e.g., activity) that the user 102 may participate in, a location 305 of an scheduled event, or both, such that the user device 106 may determine a location 305 associated with a calendar entry. For example, a calendar application may include a calendar event for “tennis” associated with a tennis center illustrated by location 305-a such that the system 300 (e.g., wearable device 104, user device 106, servers 110) is able to determine that the user is likely located at (e.g., within) the location 305-a for the duration of the “tennis” calendar event. Additionally, information from calendar events may enable the system 300 to “learn” or identify certain locations 305. For example, in cases where the calendar application executable by the user device 106 includes a calendar event associated with personal training at the given time, the system 300 may be configured to label or identify a location 305 that the user is located during the personal training event as a gym. Similarly, in some cases, the user 102 may be able to define or label semantic locations 305. For example, while located at location 305-b, the user 102 may use a user device 106 to label the location 305-b as a restaurant.

Based on acquired geographical location data, the system 300 (e.g., wearable device 104, user device 106, servers 110) may be able to identify different semantic locations 305 over time. For instance, the system 300 may be able to determine that the user was at a coffee shop (e.g., location 305-c) in the morning, at a gym (e.g., location 305-b) in the afternoon, and a restaurant (e.g., location 305-a) next to the gym in the evening. In other words, the system 300 may be configured to identify that the user 102 was located at or within the various locations 305 during different time periods.

In some aspects, a wearable device 104 associated with the user 102 may collect physiological data associated with the user. Physiological data may include, but is not limited to, heart rate data, temperature data, PPG data, blood oxygen saturation data, motion/movement data (e.g., accelerometer data), and the like. In some aspects, the system 300 may be configured to associate acquired physiological data to the respective locations 305. In other words, for physiological data that was collected/acquired while the user 102 is located at location 305-a, the system 300 may be configured to label the collected physiological data as being associated with (e.g., collected within or at) the location 305-a.

As shown in FIG. 3, at 310, the system 300 may be configured to correlate the acquired physiological data with one or more candidate taggable events. In some aspects, the one or more candidate taggable events may be selected from a set of candidate taggable events defined within an application associated with (e.g., executable by) the wearable device 104, the user device 106, or both. For example, the wearable application 250 illustrated in FIG. 2 may include or define various taggable events indicating various events, conditions, activities, etc., that the user 102 may engage in. Candidate taggable events may include, but are not limited to, a workout or exercise, food consumption, caffeine consumption, alcohol consumption, and the like.

In some aspects, the system 300 may correlate acquired physiological data with candidate taggable events based on characteristics or parameters of the physiological data. For example, in cases where the physiological data exhibits increased temperature and increased heart rate, the system 300 may correlate the physiological data with exercise and/or caffeine consumption as candidate taggable events which may be associated with (e.g., contribute to) the increased temperature and increased heart rate.

In some aspects, each candidate taggable event may be associated with a corresponding confidence value that indicates a confidence level that the corresponding candidate taggable event occurred. In other words, a confidence value may represent a relative likelihood or probability that the user 102 engaged in or experienced the corresponding taggable event. In particular, confidence values may indicate relative confidence levels that the corresponding taggable event occurred within some given time interval. For example, the user 102 may be located at location 305-a during a time interval, and the system 300 may identify a workout as a candidate taggable event that occurred during the time interval that the user 102 was at location 305-a (based on physiological data collected before, during, and/or after the time interval). In this example, the confidence value may indicate a relative probability or likelihood that the user engaged in a workout during the time interval that the user was at location 305-a.

Continuing with reference to FIG. 3, at 315, the system 300 (e.g., wearable device 104, user device 106, servers 110) may be configured to selectively modify one or more confidence values associated with the candidate taggable events to generate modified confidence values. In other words, the system 300 may adjust a prediction as to a relative likelihood or probability that the user 102-a engaged in or experienced the respective candidate taggable events. In particular, the system 300 may modify the one or more confidence values based on the geographical location data 320.

For example, in some cases, physiological data acquired from the user 102-a may indicate high temperature and/or high heart rate readings, and the system 300 may correlate the physiological data with exercise, caffeine consumption, and alcohol consumption as candidate taggable events. In this example, the system 300 may identify that the user 102-a was at a coffee shop (e.g., location 305-a) before or during the acquired physiological data that indicated high temperature and heart rate readings. As such, the system 300 may determine that it is more likely that user consumed caffeine that resulted in the high temperature/heart rate readings, and that it is less likely that the user performed a workout or consumed alcohol while at the coffee shop. Accordingly, the system 300 may selectively increase the confidence value associated with caffeine consumption, and selectively decrease the confidence values associated with alcohol consumption and exercise based on the geographical location data 320.

Subsequently, at 325, the system 300 may be configured to identify a taggable event based on one of the modified confidence values satisfying some threshold confidence value. For example, continuing with the example above, the system 300 may identify and tag “caffeine consumption” as a taggable event for the user 102-a based on the modified confidence value associated with caffeine consumption satisfying a threshold confidence value.

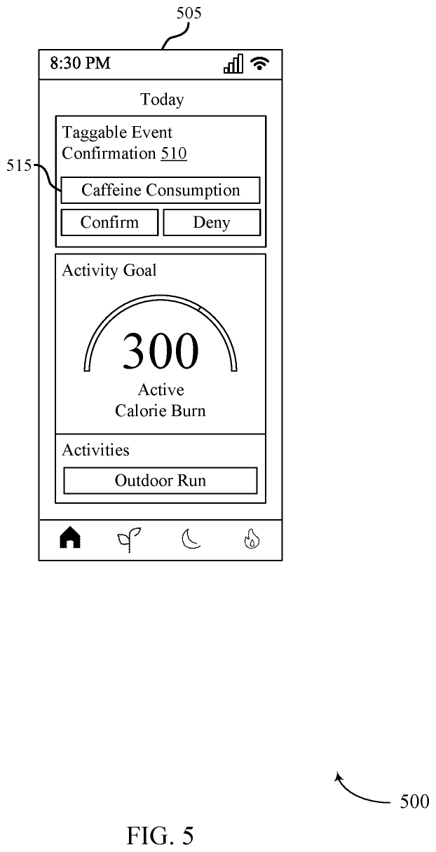

In some aspects, upon identifying a taggable event for the user, the system 300 may display information associated with the identified taggable event to the user 102-a. For example, as shown in FIG. 2, a GUI 275 of the user device 106 may display an indication of the identified taggable event. In some implementations, as will be described in further detail with respect to FIG. 5, a user may be able to confirm, deny, and/or modify the identified taggable event (e.g., confirm or deny whether the taggable event occurred, modify a time that the taggable event occurred, modify a type of taggable event, etc.).

In some aspects, the various steps/functions illustrated in FIG. 3 (and throughout the present disclosure) may be performed via a machine learning model (e.g., classifier, machine learning algorithm) executed or hosted by the wearable device 104, the user device 106, the servers 110, or any combination thereof. For example, in some implementations, the system 300 may input physiological data and the geographical location data associated with the user 102-a into a machine learning model, where the machine learning model is configured to correlate the physiological data with the one or more taggable events, generate confidence values associated with the respective confidence values, and the like. Similarly, in some implementations, the system 300 may be configured to input the geographical location data and candidate taggable events into a machine learning model, where the machine learning model is configured to selectively adjust the confidence values associated with the candidate taggable events, identify a taggable event, or both.

In some implementations, as will be described in further detail herein, users 102 may be able to confirm, deny, and/or modify identified taggable events. In such cases, such user inputs (e.g., confirmations, modifications, etc.) related to identified taggable events may be inputted into the machine learning model(s) to further train the machine learning model(s) to accurately and reliably identify taggable events. Moreover, in some implementations, machine learning models may be trained for each respective user 102 such that each respective machine learning model is “tailored” or customized to identify taggable events for the respective user. For example, a user device 106 associated with the user 102-a may train a machine learning model based on historical physiological data and/or historical taggable events identified for the user so that the machine learning model is able to accurately and reliably identify taggable events for the user 102-a.

In some aspects, identified and/or confirmed taggable events may be used to identify relationships between the user's habits and behaviors, and the user's physiological data/overall health. In other words, the system 300 may be configured to identify how certain taggable events affect the user's overall health, and generate insights/recommendations to the users 102 to help the users 102 improve their overall health. In this regard, the system 300 may be configured to identify a relationship between the identified taggable event, and physiological data acquired from the user, and display a message associated with the relationship.

For example, the system 300 may identify that the user 102-a consumed coffee in the afternoon (e.g., identify “caffeine consumption” as a taggable event). In this example, the system 300 may determine that the user 102-a exhibited heightened temperature and respiratory rates later that night that disturbed the user's 102-a sleep. In this regard, the system 300 may identify a relationship between the taggable event (e.g., caffeine consumption) and the physiological data later that night (e.g., heightened temperature/respiratory rate). The system 300 may display information associated with the identified relationship to the user 102-a so that the user can take actions to improve their health. For instance, the system 300 may display, via a GUI 275, a message that indicates late caffeine consumption may have disturbed the user's sleep, and that the user 102-a should consider limiting caffeine consumption in the afternoons to improve their sleep quality.

In some aspects, acquired physiological data may be used to identify specific “gestures” that the user 102-a engaged in, which may be used to identify candidate taggable events at 310, modify confidence values at 315, identify taggable events at 325, or any combination thereof. In particular, motion data (e.g., acceleration data) acquired via the wearable device 104 may be used to identify specific gestures, such as an eating gesture, a drinking gesture, a sports-related gesture (e.g., golf swing gesture, racket sport gesture, running or walking stride/gait gesture, weight-lifting gesture for a specific type of lift, and the like). In some aspects, gestures and/or gesture profiles associated with the respective gestures may be defined within the wearable application 250 executable by the user device 106. Such identified gestures may be used to more accurately and efficiently identify taggable events.

For example, the system 300 (e.g., wearable device 104, user device 106, servers 110) may be configured to identify motion segments within motion data collected via the wearable device 104. In this example, the system 300 may be configured to identify a gesture that the user engaged in by matching one or more motion segments to a gesture profile associated with a gesture. The gesture profile may indicate a sequence or pattern of motion data in the x, y, and z direction that is indicative of the respective gesture. Upon identifying the gesture, the system 300 may use the gesture to identify taggable events, modify confidence values, and the like. For example, in cases where the system 300 identifies a drinking gesture, the system 300 may selectively increase confidence values associated with drinking-related taggable events, such as caffeine consumption and alcohol consumption.

In some aspects, the system 300 may correlate identified gestures with the respective locations 305 in which the gestures were performed. Correlations between gestures and the respective locations 305 may be further used to identify taggable events for the user 102-a. For example, if the user is located at a gym (e.g., location 305-b), and identifies a weight-lifting gesture based on motion data collected while the user 102-a was at the gym, the system 300 may determine that it is likely the user engaged in a workout (e.g., taggable event) while at the gym. Moreover, as described previously herein, the system 300 may be configured to identify relationships between identified gestures and physiological data acquired from the user to generate insights and/or recommendations that may be used to help improve the user's overall health.

In some implementations, identified gestures and/or taggable events may be used to selectively adjust scores (e.g., Readiness Score, Activity Score, Sleep Score) associated with the user 102-a. For example, upon identifying a workout as a taggable event for the user 102-a, the system 300 may selectively adjust a Readiness Score, an Activity Score, a Sleep Score, or any combination thereof.

In some aspects, historical taggable event data associated with the user 102-a may be used to further refine and improve taggable event identification for the user 102-a. In other words, the system 300 may be configured to utilize previous taggable events, including locations where the previous taggable events occurred, to improve the efficiency and accuracy with which the system 300 is able to identify taggable events for the user.

For example, the user 102-a may frequently attend workout classes at their gym, which may be identified as location 305-a. In this example, the system 300 may store and identify historical taggable event data that includes historical taggable events (e.g., previous workouts) performed by the user 102-a while located at location 305-a. In this example, if the system 300 identifies that the user 102-a has returned to the gym (e.g., returned to location 305-a), the system 300 may be configured to determine it is likely that the user 102-a will participate in another workout while at the gym. In this regard, the system 300 may be configured to identify a workout as a potential candidate event at 310, modify a confidence value associated with a workout taggable event at 315, and/or identify a workout as a taggable event at 325 based on the historical taggable event data.

Additionally, or alternatively, the system 300 may be configured to leverage data from other users 102 (e.g., users 102-a, 102-b) and/or other devices (e.g., other wearable devices 104, other user devices 106) to improve identification of taggable events. In particular, the system 300 may be configured to identify when the user 102-a is in the proximity of other users 102-b, 102-b (e.g., in the same location 305 as other users 102) to identify candidate taggable events, modify confidence values 315, identify taggable events 325, and the like.

For example, in some aspects, the system 300 may determine that the user 102-a is at the semantic location 305-a, and that one or more additional users 102-b, 102-c are also located at the semantic location 305-a at the same time as the user 102-a. In some cases, a wearable device 104 and/or user device 106 associated with the user 102-a may communicate with respective devices associated with the additional users 102-b, 102-c to determine that the user 102-a is likely in the same semantic location 305-a as the additional users 102-b, 102-c. Moreover, in some cases, the user 102-a may be able to define or label additional users 102 (or devices associated with the respective additional users 102-b, 102-c) within the wearable application 250, as well as relationships of the additional users 102 with respect to the user 102-a.

Continuing with the same example, the system 300 may use the information regarding the additional semantic users 102 to identify the candidate taggable events at 310, modify the confidence values at 315, identify the taggable event at 325, or any combination thereof. For example, if the user 102-a has frequently consumed alcohol at a bar (e.g., semantic location 305-a) with the additional users 102-b, 102-c in the past, and the user 102-a has returned to the bar with the additional users 102-b, 102-c, the system 300 may determine that it is likely that the user 102-a has (or will) consume alcohol while at the bar. As such, at 315, the system 300 may selectively increase a confidence value associated with alcohol consumption.

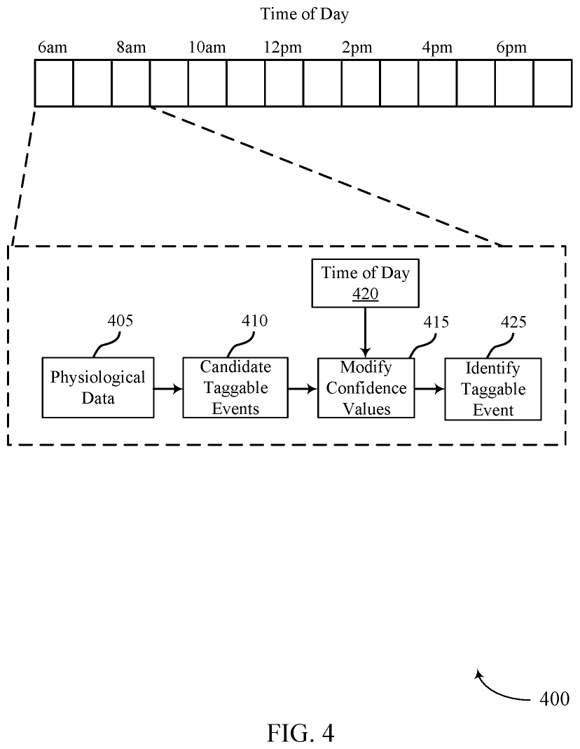

FIG. 4 illustrates an example of a timeline 400 that supports techniques for using a hybrid model for generating tags and insights in accordance with aspects of the present disclosure. The timeline 400 may implement, or be implemented by, aspects of the system 100, the system 200, the system 300, or any combination thereof.

In some aspects, the timeline 400 shown and described in FIG. 4 illustrates how the techniques described herein may identify a relative probability or likelihood of certain taggable events taking place based on the time of day. The techniques shown and described in FIG. 4 may be used in addition to, or in the alternate to, the geographical location techniques shown and described in FIG. 3. In this regard, any description associated with FIG. 3 may be regarded as applying to the techniques shown and described in FIG. 4, to the extent applicable.

As shown in FIG. 4, in some aspects, a wearable device 104 may acquire physiological data from a user at 405. Physiological data may include, but is not limited to, heart rate data, temperature data, PPG data, blood oxygen saturation data, motion/movement data (e.g., accelerometer data), and the like. In some aspects, acquired physiological data may be time stamped to indicate the time of day in which the respective physiological measurements were performed.

As described previously herein, at 410, the system may be configured to correlate the acquired physiological data with one or more candidate taggable events. In some aspects, the one or more candidate taggable events may be selected from a set of candidate taggable events defined within an application associated with (e.g., executable by) the wearable device 104, the user device 106, or both. In some aspects, each candidate taggable event may be associated with a corresponding confidence value that indicates a confidence level that the corresponding candidate taggable event occurred. In other words, a confidence value may represent a relative likelihood or probability that the user 102 engaged in or experienced the corresponding taggable event. In particular, confidence values may indicate relative confidence levels that the corresponding taggable event occurred within some given time interval.

For example, as shown in FIG. 4, the system may identify candidate taggable events within the time interval between 6 am and 8 am, where the confidence values indicate relative likelihoods or probabilities that the candidate taggable events occurred within the time interval. In some aspects, the system may identify the candidate taggable events based on physiological data collected before, during, and/or after the time interval.

For example, motion data collected during the time interval between 6 am and Sam may be used to determine that the user likely performed a workout in that time (e.g., exercise taggable event). By way of another example, elevated heart rate readings collected later in the morning (e.g., between 9 am and 10 am) may suggest that the user consumed coffee during the time interval between 6 am and 8 am.

Continuing with reference to FIG. 4, at 415, the system (e.g., wearable device 104, user device 106, servers 110) may be configured to selectively modify one or more confidence values associated with the candidate taggable events to generate modified confidence values. In other words, the system may adjust a prediction as to a relative likelihood or probability that the user engaged in or experienced the respective candidate taggable events. In particular, the system may modify the one or more confidence values based on time of day 420 (e.g., 6 am to 8 am) that the candidate taggable events have been identified.

For example, in some cases, physiological data acquired from the user 102 during the time interval between 6 am and Sam may indicate high temperature and/or high heart rate readings, and the system may correlate the physiological data with caffeine consumption and alcohol consumption as candidate taggable events. In this example, the system may determine that it is more likely that the user consumed caffeine that resulted in the high temperature/heart rate readings, and that it is less likely that the user consumed alcohol this early in the morning (e.g., based on the time of day 420). Accordingly, the system may selectively increase the confidence value associated with caffeine consumption, and selectively decrease the confidence values associated with alcohol consumption based on the time of day 420.

Comparatively, consider the same example, but where the candidate taggable events are identified in the late evening as opposed to the early morning. In this example, physiological data acquired from the user 102 during a time interval between 8 pm and 10 pm may once again indicate high temperature and/or high heart rate readings, and the system may again correlate the physiological data with caffeine consumption and alcohol consumption as candidate taggable events. In this example, the system may determine that it is more likely that the user consumed alcohol (rather than caffeine) due to the time of day 420 that the candidate taggable events were identified (e.g., 9 pm to 10 pm). Accordingly, the system may selectively increase the confidence value associated with alcohol consumption, and selectively decrease the confidence values associated with caffeine consumption based on the time of day 420.

Subsequently, at 425, the system may be configured to identify a taggable event based on one of the modified confidence values satisfying some threshold confidence value. For example, continuing with the example above, the system may identify and tag “alcohol consumption” as a taggable event for the user based on the modified confidence value associated with alcohol consumption satisfying a threshold confidence value.

In some aspects, historical taggable event data associated with a relative timing (e.g., time of day 420) that the user performs or engages in certain taggable events may be used to further improve identification of taggable events. In other words, the system may be configured to utilize previous taggable events, including relative times of day 420 when the previous taggable events occurred, to improve the efficiency and accuracy with which the system is able to identify taggable events for the user.

For example, a user may drink coffee at work every weekday during the time interval between Sam and 10 am. In this example, the system may store and identify historical taggable event data that includes historical taggable events (e.g., previous caffeine consumption) performed by the user during weekdays between Sam and 10 am. In this example, during subsequent weekdays, the system may selectively increase confidence values associated with caffeine consumption during weekdays between Sam and 10 am. In other words, the system may identify that it is more likely that the user will (or has) consumed caffeine between 6 am and Sam based on the time of day 420 and historical taggable events identified within the respective time of day 420.

FIG. 5 illustrates an example of a GUI 500 that supports techniques for using a hybrid model for generating tags and insights in accordance with aspects of the present disclosure. The GUI 500 may implement, or be implemented by, aspects of the system 100, the system 200, the system 300, the timeline 400, or any combination thereof. For example, the GUI 500 may include an example of the GUI included within a user device 106.

The GUI 500 illustrates a series of application pages 505 that may be displayed to the user via the GUI 500 (e.g., GUI 275 illustrated in FIG. 2). Continuing with the example above, the ring 104 may identify a taggable event 515 that the user 102 engaged or participated in based on collected physiological data and additional types/sources of data, such as geographical location data 320, data associated with a time of day 420, historical taggable event data, proximity to additional users, and the like.

Upon identifying the taggable event 515, the user 102 may be presented with the application page 505-a. As shown in FIG. 5, the application page 505-a may display an indication that the taggable event 515. In some cases, the indication may include a taggable event confirmation 510 that may prompt the user to confirm or dismiss the taggable event 515 (e.g., confirm/deny whether the system correctly identified the taggable event 515). Additionally, or alternatively, the taggable event confirmation 510 may display a confirmation message to the user indicating that the taggable event 515 was successfully recorded (e.g., automatically record the taggable event 515 without user confirmation).

In some aspects, a user input received via the taggable event confirmation 510 may be used to further train a classifier, machine learning model, or other algorithm that is configured to perform taggable event recognition. In particular, a user input that confirms or denies an identified taggable event may be inputted back into the classifier to further train the classifier for performing taggable event recognition.

In some implementations, the taggable event 515 may be recorded/logged in a database. For example, the taggable event 515 may be logged in an activity log for the user for the respective day. In some aspects, the system may associate the taggable event 515 with physiological data collected from the user 102 (e.g., during the time interval that the taggable event 515 was recorded or during a different time interval) in order to identify trends or relationships between taggable events 515 and the user's physiological data (e.g., determine that consuming caffeine after 7 pm leads to poor quality sleep for the user). For example, if the system identifies alcohol as a taggable event for the user between 10 μm and midnight, and subsequently identifies that the user suffered from poor sleep, the system may determine that consuming alcohol late at night leads to poor quality sleep for the user. In such cases, the application pages 505 may display insights or recommendations based on the identified relationship in order to help improve the user's overall health (e.g., recommend that the user limit their alcohol consumption before bed to improve their sleep quality).

Moreover, in some cases, the taggable event 515 may be used to update (e.g., modify) one or more scores associated with the user (e.g., Sleep Score, Readiness Score). That is, data associated with the taggable event 515 may be used to update the scores for the user for the respective day that taggable event 515 was detected. In some cases, the application page 505-a may display the one or more scores for the user for the respective day.

In some implementations, the system may be configured to log, record, or otherwise recognize data associated with a taggable event 515 without explicit confirmation from a user 102. For example, in some cases, the system may identify the taggable event 515 with a sufficient degree of precision, accuracy, or reliability. In such cases, the system may log or otherwise record the taggable event 515 without displaying a prompt (e.g., the taggable event confirmation 510) to a user 102 and/or receiving an explicit confirmation from the user 120.

In cases where the user 102 dismisses (e.g., denies) the taggable event confirmation 510 (e.g., prompt) on the application page 505-a, the taggable event confirmation 510 may disappear, and the data associated with the (incorrect) taggable event confirmation 510 may not be used to update the user's 102 scores or logged in an activity log for the user 102 for the respective day. Moreover, in some implementations, the user may be able to selectively modify one or more parameters or characteristics associated with the taggable event 515, such as an estimated time that the taggable event 515 occurred, a type of taggable event, and the like.

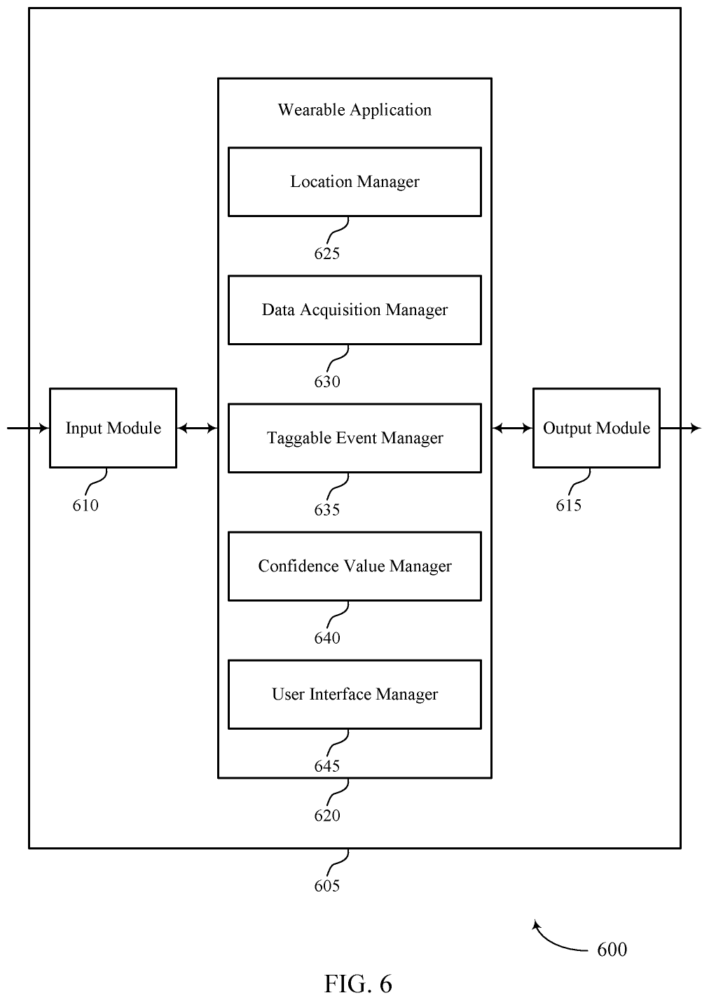

FIG. 6 shows a block diagram 600 of a device 605 that supports techniques for using a hybrid model for generating tags and insights in accordance with aspects of the present disclosure. The device 605 may include an input module 610, an output module 615, and a wearable application 620. The device 605 may also include a processor. Each of these components may be in communication with one another (e.g., via one or more buses).

The input module 610 may provide a means for receiving information such as packets, user data, control information, or any combination thereof associated with various information channels (e.g., control channels, data channels, information channels related to illness detection techniques). Information may be passed on to other components of the device 605. The input module 610 may utilize a single antenna or a set of multiple antennas.

The output module 615 may provide a means for transmitting signals generated by other components of the device 605. For example, the output module 615 may transmit information such as packets, user data, control information, or any combination thereof associated with various information channels (e.g., control channels, data channels, information channels related to illness detection techniques). In some examples, the output module 615 may be co-located with the input module 610 in a transceiver module. The output module 615 may utilize a single antenna or a set of multiple antennas.

For example, the wearable application 620 may include a location manager 625, a data acquisition manager 630, a taggable event manager 635, a confidence value manager 640, a user interface manager 645, or any combination thereof. In some examples, the wearable application 620, or various components thereof, may be configured to perform various operations (e.g., receiving, monitoring, transmitting) using or otherwise in cooperation with the input module 610, the output module 615, or both. For example, the wearable application 620 may receive information from the input module 610, send information to the output module 615, or be integrated in combination with the input module 610, the output module 615, or both to receive information, transmit information, or perform various other operations as described herein.

The wearable application 620 may support identifying taggable events using a wearable device in accordance with examples as disclosed herein. The location manager 625 may be configured as or otherwise support a means for receiving geographical location data associated with a user throughout a time interval. The data acquisition manager 630 may be configured as or otherwise support a means for receiving physiological data associated with the user from a wearable device. The taggable event manager 635 may be configured as or otherwise support a means for correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval. The confidence value manager 640 may be configured as or otherwise support a means for selectively modifying the one or more confidence values associated with the one or more candidate taggable events based at least in part on the geographical location data to generate one or more modified confidence values. The taggable event manager 635 may be configured as or otherwise support a means for identifying a taggable event of the one or more candidate taggable events within the time interval based at least in part on a modified confidence value associated with the taggable event satisfying a threshold confidence value. The user interface manager 645 may be configured as or otherwise support a means for causing a GUI of a user device running the application to display an indication of the identified taggable event.

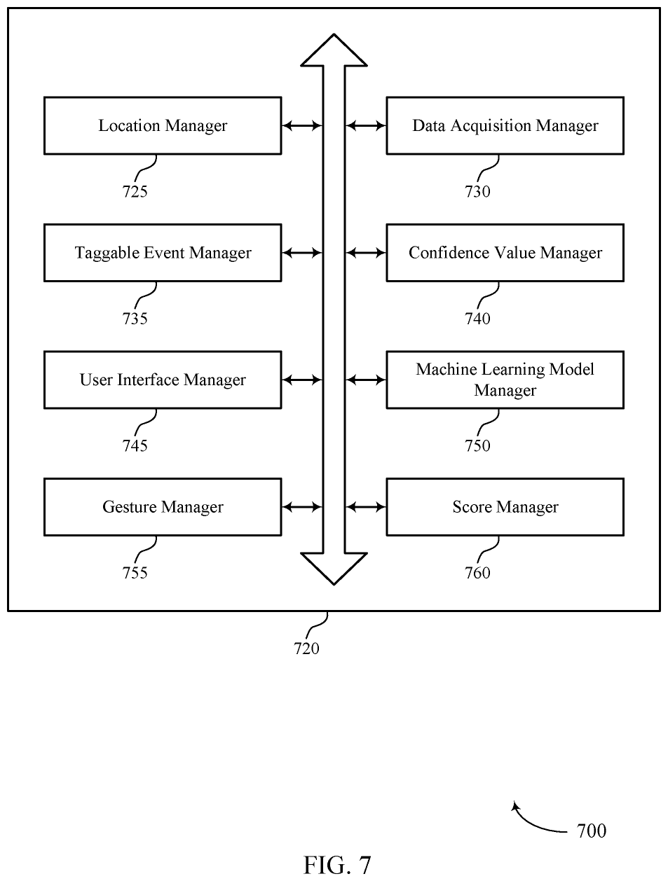

FIG. 7 shows a block diagram 700 of a wearable application 720 that supports techniques for using a hybrid model for generating tags and insights in accordance with aspects of the present disclosure. The wearable application 720 may be an example of aspects of a wearable application or a wearable application 620, or both, as described herein. The wearable application 720, or various components thereof, may be an example of means for performing various aspects of techniques for using a hybrid model for generating tags and insights as described herein. For example, the wearable application 720 may include a location manager 725, a data acquisition manager 730, a taggable event manager 735, a confidence value manager 740, a user interface manager 745, a machine learning model manager 750, a gesture manager 755, a score manager 760, or any combination thereof. Each of these components may communicate, directly or indirectly, with one another (e.g., via one or more buses).

The wearable application 720 may support identifying taggable events using a wearable device in accordance with examples as disclosed herein. The location manager 725 may be configured as or otherwise support a means for receiving geographical location data associated with a user throughout a time interval. The data acquisition manager 730 may be configured as or otherwise support a means for receiving physiological data associated with the user from a wearable device. The taggable event manager 735 may be configured as or otherwise support a means for correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval. The confidence value manager 740 may be configured as or otherwise support a means for selectively modifying the one or more confidence values associated with the one or more candidate taggable events based at least in part on the geographical location data to generate one or more modified confidence values. In some examples, the taggable event manager 735 may be configured as or otherwise support a means for identifying a taggable event of the one or more candidate taggable events within the time interval based at least in part on a modified confidence value associated with the taggable event satisfying a threshold confidence value. The user interface manager 745 may be configured as or otherwise support a means for causing a GUI of a user device running the application to display an indication of the identified taggable event.

In some examples, the taggable event manager 735 may be configured as or otherwise support a means for identifying a relationship between the identified taggable event and the physiological data acquired during the time interval, additional physiological data acquired during a different time interval, or both. In some examples, the user interface manager 745 may be configured as or otherwise support a means for causing the GUI of the user device to display a message associated with the relationship.

In some examples, the machine learning model manager 750 may be configured as or otherwise support a means for inputting the physiological data and the geographical location data into a machine learning model, wherein correlating the physiological data with the one or more candidate taggable events, selectively modifying the one or more confidence values, identifying the taggable event, or any combination thereof, is based at least in part on inputting the physiological data and the geographical location data into a machine learning model.

In some examples, the taggable event manager 735 may be configured as or otherwise support a means for identifying historical taggable event data associated with the user, the historical taggable event data comprising a plurality of historical taggable events identified for the user and historical geographical location data corresponding to the plurality of historical taggable events. In some examples, the location manager 725 may be configured as or otherwise support a means for identifying that the geographical location data of the user is associated with the historical geographical location data, wherein selectively modifying the one or more confidence values is based at least in part on the historical taggable event data and identifying that the geographical location data of the user is associated with the historical geographical location data.

In some examples, the taggable event manager 735 may be configured as or otherwise support a means for identifying historical taggable event data associated with the user, the historical taggable event data comprising a plurality of historical taggable events and a time of day in which the plurality of historical taggable events were identified. In some examples, the confidence value manager 740 may be configured as or otherwise support a means for identifying that the time interval during which the physiological data was acquired is within the time of day, wherein selectively modifying the one or more confidence values is based at least in part on the historical taggable event data and identifying that the time interval is within the time of day.

In some examples, the user interface manager 745 may be configured as or otherwise support a means for receiving, via the GUI and based at least in part on displaying the indication of the taggable event, a confirmation of the taggable event, a modification of the taggable event, or both.

In some examples, the physiological data comprises at least motion data, and the data acquisition manager 730 may be configured as or otherwise support a means for identifying a plurality of motion segments within the time interval based at least in part on the motion data. In some examples, the physiological data comprises at least motion data, and the gesture manager 755 may be configured as or otherwise support a means for identifying a gesture the user engaged in based at least in part on matching a motion segment of the plurality of motion segments to a gesture profile of a set of gesture profiles defined within the application, wherein selectively modifying the one or more confidence values, identifying the taggable event, or both, is based at least in part on identifying the gesture.

In some examples, the gesture manager 755 may be configured as or otherwise support a means for identifying a relationship between the identified gesture and the physiological data acquired during the time interval, additional physiological data acquired during a different time interval, or both, wherein correlating the physiological data with one or more candidate taggable events, selectively modifying the one or more confidence values, identifying the taggable event, or any combination thereof, is based at least in part on identifying the relationship.

In some examples, the geographical location data is associated with a semantic location, and the location manager 725 may be configured as or otherwise support a means for determining that one or more additional users are located at the semantic location during at least a portion of the time interval, wherein selectively modifying the one or more confidence values, identifying the taggable event, or both, is based at least in part on determining that one or more additional users are located at the semantic location during at least a portion of the time interval.

In some examples, the score manager 760 may be configured as or otherwise support a means for selectively adjusting a Readiness Score associated with the user, an Activity Score associated with the user, a Sleep Score associated with the user, or any combination thereof, based at least in part on the identified taggable event.

In some examples, the geographical location data comprises geographical positioning data acquired via the user device. In some examples, the geographical location data is received via a calendar application executable by the user device. In some examples, the taggable event comprises a workout, food consumption, caffeine consumption, alcohol consumption, or any combination thereof.

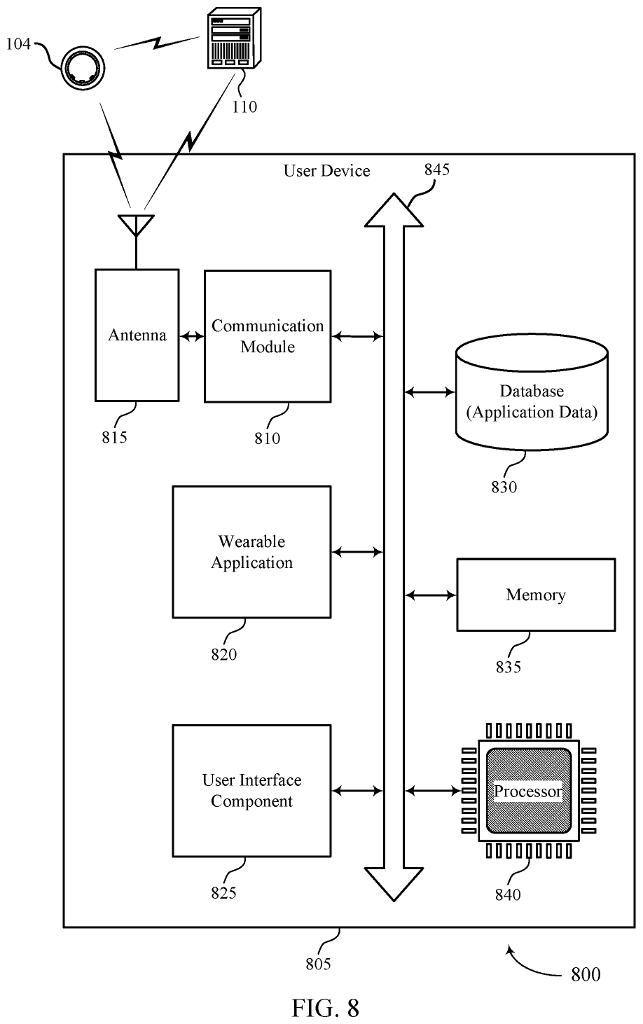

FIG. 8 shows a diagram of a system 800 including a device 805 that supports techniques for using a hybrid model for generating tags and insights in accordance with aspects of the present disclosure. The device 805 may be an example of or include the components of a device 605 as described herein. The device 805 may include an example of a user device 106, as described previously herein. The device 805 may include components for bi-directional communications including components for transmitting and receiving communications with a wearable device 104 and a server 110, such as a wearable application 820, a communication module 810, an antenna 815, a user interface component 825, a database (application data) 830, a memory 835, and a processor 840. These components may be in electronic communication or otherwise coupled (e.g., operatively, communicatively, functionally, electronically, electrically) via one or more buses (e.g., a bus 845).

The communication module 810 may manage input and output signals for the device 805 via the antenna 815. The communication module 810 may include an example of the communication module 220-b of the user device 106 shown and described in FIG. 2. In this regard, the communication module 810 may manage communications with the ring 104 and the server 110, as illustrated in FIG. 2. The communication module 810 may also manage peripherals not integrated into the device 805. In some cases, the communication module 810 may represent a physical connection or port to an external peripheral. In some cases, the communication module 810 may utilize an operating system such as iOS®, ANDROID®, MS-DOS®, MS-WINDOWS®, OS/2®, UNIX®, LINUX®, or another known operating system. In other cases, the communication module 810 may represent or interact with a wearable device (e.g., ring 104), modem, a keyboard, a mouse, a touchscreen, or a similar device. In some cases, the communication module 810 may be implemented as part of the processor 840. In some examples, a user may interact with the device 805 via the communication module 810, user interface component 825, or via hardware components controlled by the communication module 810.

In some cases, the device 805 may include a single antenna 815. However, in some other cases, the device 805 may have more than one antenna 815, which may be capable of concurrently transmitting or receiving multiple wireless transmissions. The communication module 810 may communicate bi-directionally, via the one or more antennas 815, wired, or wireless links as described herein. For example, the communication module 810 may represent a wireless transceiver and may communicate bi-directionally with another wireless transceiver. The communication module 810 may also include a modem to modulate the packets, to provide the modulated packets to one or more antennas 815 for transmission, and to demodulate packets received from the one or more antennas 815.

The user interface component 825 may manage data storage and processing in a database 830. In some cases, a user may interact with the user interface component 825. In other cases, the user interface component 825 may operate automatically without user interaction. The database 830 may be an example of a single database, a distributed database, multiple distributed databases, a data store, a data lake, or an emergency backup database.

The memory 835 may include RAM and ROM. The memory 835 may store computer-readable, computer-executable software including instructions that, when executed, cause the processor 840 to perform various functions described herein. In some cases, the memory 835 may contain, among other things, a BIOS which may control basic hardware or software operation such as the interaction with peripheral components or devices.

The processor 840 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 840 may be configured to operate a memory array using a memory controller. In other cases, a memory controller may be integrated into the processor 840. The processor 840 may be configured to execute computer-readable instructions stored in a memory 835 to perform various functions (e.g., functions or tasks supporting a method and system for sleep staging algorithms).

The wearable application 820 may support identifying taggable events using a wearable device in accordance with examples as disclosed herein. For example, the wearable application 820 may be configured as or otherwise support a means for receiving geographical location data associated with a user throughout a time interval. The wearable application 820 may be configured as or otherwise support a means for receiving physiological data associated with the user from a wearable device. The wearable application 820 may be configured as or otherwise support a means for correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval. The wearable application 820 may be configured as or otherwise support a means for selectively modifying the one or more confidence values associated with the one or more candidate taggable events based at least in part on the geographical location data to generate one or more modified confidence values. The wearable application 820 may be configured as or otherwise support a means for identifying a taggable event of the one or more candidate taggable events within the time interval based at least in part on a modified confidence value associated with the taggable event satisfying a threshold confidence value. The wearable application 820 may be configured as or otherwise support a means for causing a GUI of a user device running the application to display an indication of the identified taggable event.

The wearable application 820 may include an application (e.g., “app”), program, software, or other component which is configured to facilitate communications with a ring 104, server 110, other user devices 106, and the like. For example, the wearable application 820 may include an application executable on a user device 106 which is configured to receive data (e.g., physiological data) from a ring 104, perform processing operations on the received data, transmit and receive data with the servers 110, and cause presentation of data to a user 102.

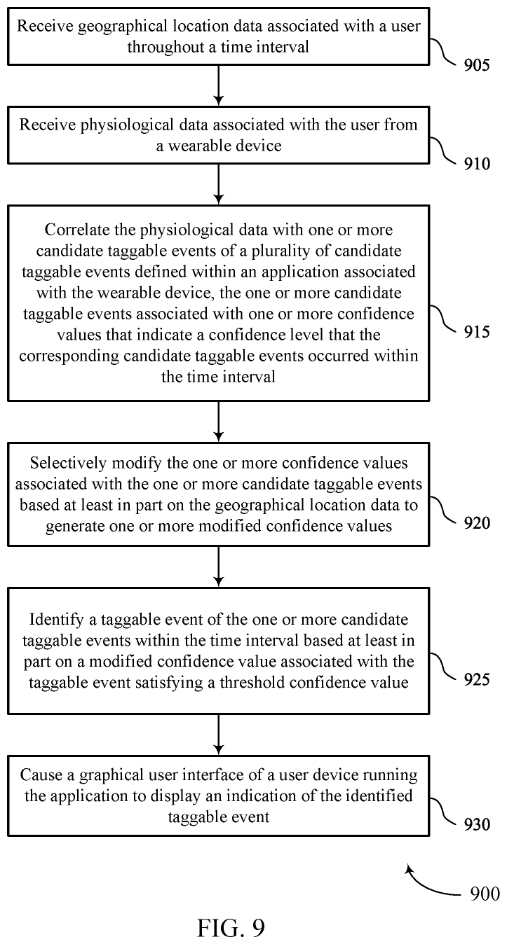

FIG. 9 shows a flowchart illustrating a method 900 that supports techniques for using a hybrid model for generating tags and insights in accordance with aspects of the present disclosure. The operations of the method 900 may be implemented by a user device or its components as described herein. For example, the operations of the method 900 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 905, the method may include receiving geographical location data associated with a user throughout a time interval. The operations of 905 may be performed in accordance with examples as disclosed herein. In some examples, aspects of the operations of 905 may be performed by a location manager 725 as described with reference to FIG. 7.

At 910, the method may include receiving physiological data associated with the user from a wearable device. The operations of 910 may be performed in accordance with examples as disclosed herein. In some examples, aspects of the operations of 910 may be performed by a data acquisition manager 730 as described with reference to FIG. 7.

At 915, the method may include correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval. The operations of 915 may be performed in accordance with examples as disclosed herein. In some examples, aspects of the operations of 915 may be performed by a taggable event manager 735 as described with reference to FIG. 7.

At 920, the method may include selectively modifying the one or more confidence values associated with the one or more candidate taggable events based at least in part on the geographical location data to generate one or more modified confidence values. The operations of 920 may be performed in accordance with examples as disclosed herein. In some examples, aspects of the operations of 920 may be performed by a confidence value manager 740 as described with reference to FIG. 7.

At 925, the method may include identifying a taggable event of the one or more candidate taggable events within the time interval based at least in part on a modified confidence value associated with the taggable event satisfying a threshold confidence value. The operations of 925 may be performed in accordance with examples as disclosed herein. In some examples, aspects of the operations of 925 may be performed by a taggable event manager 735 as described with reference to FIG. 7.

At 930, the method may include causing a GUI of a user device running the application to display an indication of the identified taggable event. The operations of 930 may be performed in accordance with examples as disclosed herein. In some examples, aspects of the operations of 930 may be performed by a user interface manager 745 as described with reference to FIG. 7.

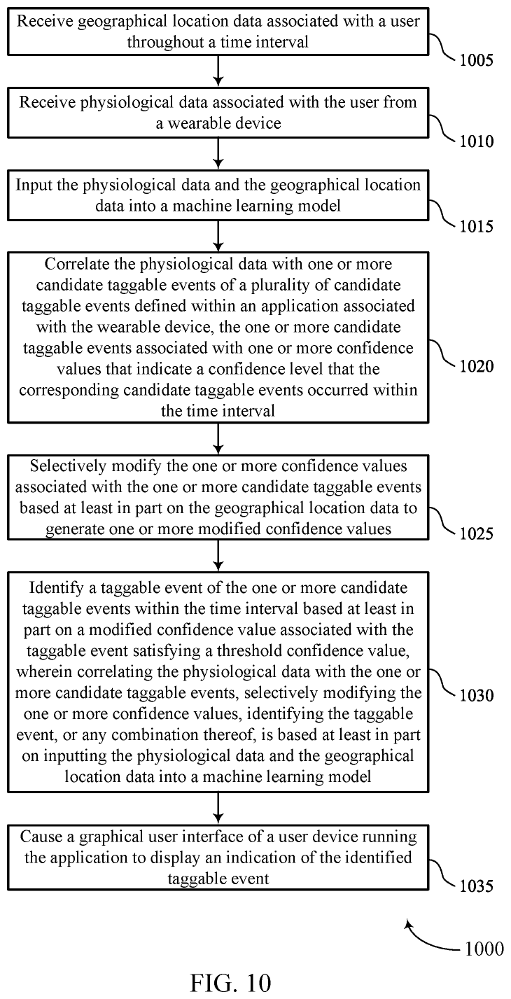

FIG. 10 shows a flowchart illustrating a method 1000 that supports techniques for using a hybrid model for generating tags and insights in accordance with aspects of the present disclosure. The operations of the method 1000 may be implemented by a user device or its components as described herein. For example, the operations of the method 1000 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 1005, the method may include receiving geographical location data associated with a user throughout a time interval. The operations of 1005 may be performed in accordance with examples as disclosed herein. In some examples, aspects of the operations of 1005 may be performed by a location manager 725 as described with reference to FIG. 7.

At 1010, the method may include receiving physiological data associated with the user from a wearable device. The operations of 1010 may be performed in accordance with examples as disclosed herein. In some examples, aspects of the operations of 1010 may be performed by a data acquisition manager 730 as described with reference to FIG. 7.

At 1015, the method may include inputting the physiological data and the geographical location data into a machine learning model, wherein correlating the physiological data with the one or more candidate taggable events, selectively modifying the one or more confidence values, identifying the taggable event, or any combination thereof, is based at least in part on inputting the physiological data and the geographical location data into a machine learning model. The operations of 1015 may be performed in accordance with examples as disclosed herein. In some examples, aspects of the operations of 1015 may be performed by a machine learning model manager 750 as described with reference to FIG. 7.

At 1020, the method may include correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval. The operations of 1020 may be performed in accordance with examples as disclosed herein. In some examples, aspects of the operations of 1020 may be performed by a taggable event manager 735 as described with reference to FIG. 7.

At 1025, the method may include selectively modifying the one or more confidence values associated with the one or more candidate taggable events based at least in part on the geographical location data to generate one or more modified confidence values. The operations of 1025 may be performed in accordance with examples as disclosed herein. In some examples, aspects of the operations of 1025 may be performed by a confidence value manager 740 as described with reference to FIG. 7.

At 1030, the method may include identifying a taggable event of the one or more candidate taggable events within the time interval based at least in part on a modified confidence value associated with the taggable event satisfying a threshold confidence value. The operations of 1030 may be performed in accordance with examples as disclosed herein. In some examples, aspects of the operations of 1030 may be performed by a taggable event manager 735 as described with reference to FIG. 7.

At 1035, the method may include causing a GUI of a user device running the application to display an indication of the identified taggable event. The operations of 1035 may be performed in accordance with examples as disclosed herein. In some examples, aspects of the operations of 1035 may be performed by a user interface manager 745 as described with reference to FIG. 7.

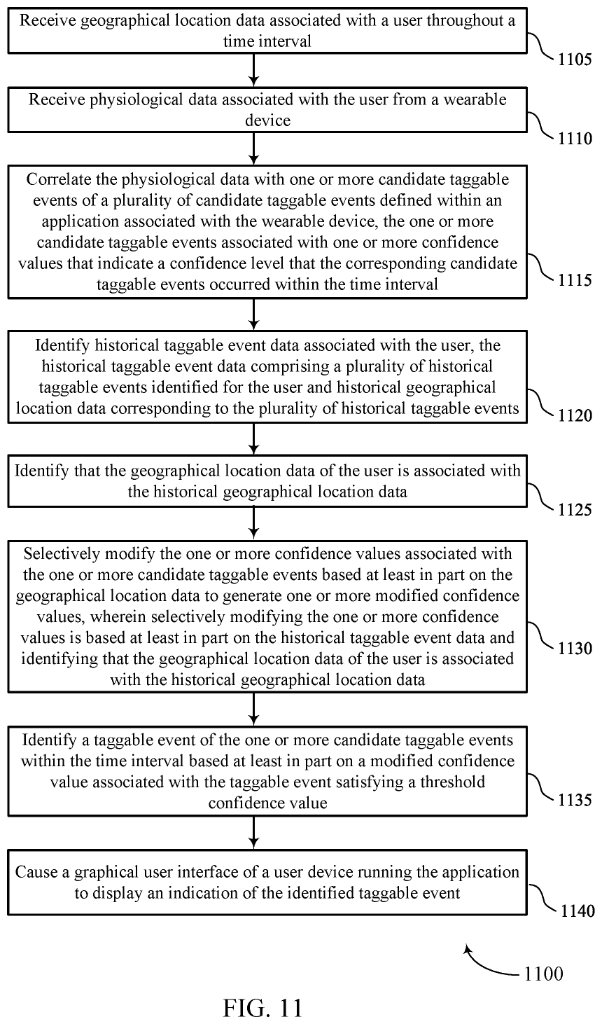

FIG. 11 shows a flowchart illustrating a method 1100 that supports techniques for using a hybrid model for generating tags and insights in accordance with aspects of the present disclosure. The operations of the method 1100 may be implemented by a user device or its components as described herein. For example, the operations of the method 1100 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 1105, the method may include receiving geographical location data associated with a user throughout a time interval. The operations of 1105 may be performed in accordance with examples as disclosed herein. In some examples, aspects of the operations of 1105 may be performed by a location manager 725 as described with reference to FIG. 7.

At 1110, the method may include receiving physiological data associated with the user from a wearable device. The operations of 1110 may be performed in accordance with examples as disclosed herein. In some examples, aspects of the operations of 1110 may be performed by a data acquisition manager 730 as described with reference to FIG. 7.

At 1115, the method may include correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval. The operations of 1115 may be performed in accordance with examples as disclosed herein. In some examples, aspects of the operations of 1115 may be performed by a taggable event manager 735 as described with reference to FIG. 7.

At 1120, the method may include identifying historical taggable event data associated with the user, the historical taggable event data comprising a plurality of historical taggable events identified for the user and historical geographical location data corresponding to the plurality of historical taggable events. The operations of 1120 may be performed in accordance with examples as disclosed herein. In some examples, aspects of the operations of 1120 may be performed by a taggable event manager 735 as described with reference to FIG. 7.

At 1125, the method may include identifying that the geographical location data of the user is associated with the historical geographical location data, wherein selectively modifying the one or more confidence values is based at least in part on the historical taggable event data and identifying that the geographical location data of the user is associated with the historical geographical location data. The operations of 1125 may be performed in accordance with examples as disclosed herein. In some examples, aspects of the operations of 1125 may be performed by a location manager 725 as described with reference to FIG. 7.

At 1130, the method may include selectively modifying the one or more confidence values associated with the one or more candidate taggable events based at least in part on the geographical location data to generate one or more modified confidence values. The operations of 1130 may be performed in accordance with examples as disclosed herein. In some examples, aspects of the operations of 1130 may be performed by a confidence value manager 740 as described with reference to FIG. 7.

At 1135, the method may include identifying a taggable event of the one or more candidate taggable events within the time interval based at least in part on a modified confidence value associated with the taggable event satisfying a threshold confidence value. The operations of 1135 may be performed in accordance with examples as disclosed herein. In some examples, aspects of the operations of 1135 may be performed by a taggable event manager 735 as described with reference to FIG. 7.

At 1140, the method may include causing a GUI of a user device running the application to display an indication of the identified taggable event. The operations of 1140 may be performed in accordance with examples as disclosed herein. In some examples, aspects of the operations of 1140 may be performed by a user interface manager 745 as described with reference to FIG. 7.

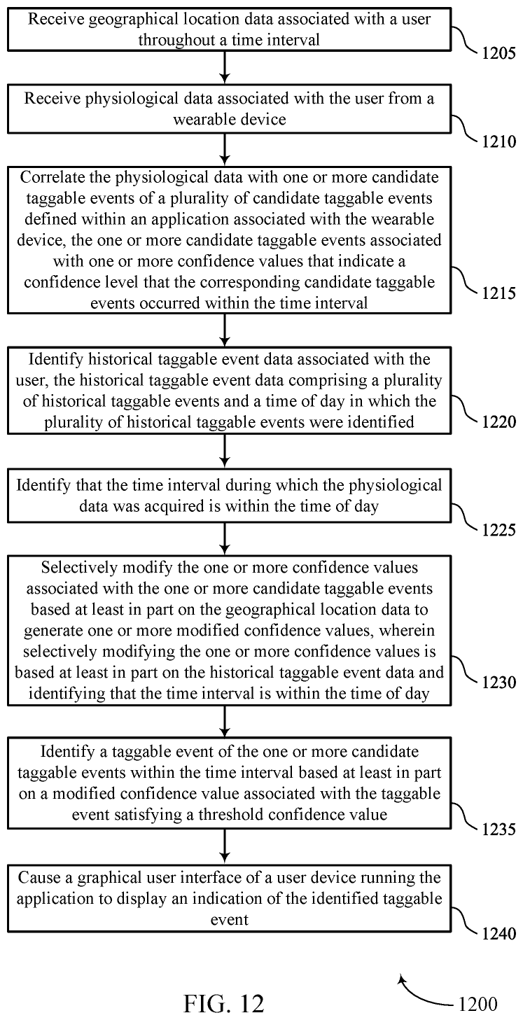

FIG. 12 shows a flowchart illustrating a method 1200 that supports techniques for using a hybrid model for generating tags and insights in accordance with aspects of the present disclosure. The operations of the method 1200 may be implemented by a user device or its components as described herein. For example, the operations of the method 1200 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 1205, the method may include receiving geographical location data associated with a user throughout a time interval. The operations of 1205 may be performed in accordance with examples as disclosed herein. In some examples, aspects of the operations of 1205 may be performed by a location manager 725 as described with reference to FIG. 7.

At 1210, the method may include receiving physiological data associated with the user from a wearable device. The operations of 1210 may be performed in accordance with examples as disclosed herein. In some examples, aspects of the operations of 1210 may be performed by a data acquisition manager 730 as described with reference to FIG. 7.

At 1215, the method may include correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval. The operations of 1215 may be performed in accordance with examples as disclosed herein. In some examples, aspects of the operations of 1215 may be performed by a taggable event manager 735 as described with reference to FIG. 7.

At 1220, the method may include identifying historical taggable event data associated with the user, the historical taggable event data comprising a plurality of historical taggable events and a time of day in which the plurality of historical taggable events were identified. The operations of 1220 may be performed in accordance with examples as disclosed herein. In some examples, aspects of the operations of 1220 may be performed by a taggable event manager 735 as described with reference to FIG. 7.

At 1225, the method may include identifying that the time interval during which the physiological data was acquired is within the time of day, wherein selectively modifying the one or more confidence values is based at least in part on the historical taggable event data and identifying that the time interval is within the time of day. The operations of 1225 may be performed in accordance with examples as disclosed herein. In some examples, aspects of the operations of 1225 may be performed by a confidence value manager 740 as described with reference to FIG. 7.

At 1230, the method may include selectively modifying the one or more confidence values associated with the one or more candidate taggable events based at least in part on the geographical location data to generate one or more modified confidence values. The operations of 1230 may be performed in accordance with examples as disclosed herein. In some examples, aspects of the operations of 1230 may be performed by a confidence value manager 740 as described with reference to FIG. 7.

At 1235, the method may include identifying a taggable event of the one or more candidate taggable events within the time interval based at least in part on a modified confidence value associated with the taggable event satisfying a threshold confidence value. The operations of 1235 may be performed in accordance with examples as disclosed herein. In some examples, aspects of the operations of 1235 may be performed by a taggable event manager 735 as described with reference to FIG. 7.

At 1240, the method may include causing a GUI of a user device running the application to display an indication of the identified taggable event. The operations of 1240 may be performed in accordance with examples as disclosed herein. In some examples, aspects of the operations of 1240 may be performed by a user interface manager 745 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method for identifying taggable events using a wearable device is described. The method may include receiving geographical location data associated with a user throughout a time interval, receiving physiological data associated with the user from a wearable device, correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval, selectively modifying the one or more confidence values associated with the one or more candidate taggable events based at least in part on the geographical location data to generate one or more modified confidence values, identifying a taggable event of the one or more candidate taggable events within the time interval based at least in part on a modified confidence value associated with the taggable event satisfying a threshold confidence value, and causing a GUI of a user device running the application to display an indication of the identified taggable event.

An apparatus for identifying taggable events using a wearable device is described. The apparatus may include a processor, memory coupled with the processor, and instructions stored in the memory. The instructions may be executable by the processor to cause the apparatus to receive geographical location data associated with a user throughout a time interval, receive physiological data associated with the user from a wearable device, correlate the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval, selectively modify the one or more confidence values associated with the one or more candidate taggable events based at least in part on the geographical location data to generate one or more modified confidence values, identify a taggable event of the one or more candidate taggable events within the time interval based at least in part on a modified confidence value associated with the taggable event satisfying a threshold confidence value, and cause a GUI of a user device running the application to display an indication of the identified taggable event.

Another apparatus for identifying taggable events using a wearable device is described. The apparatus may include means for receiving geographical location data associated with a user throughout a time interval, means for receiving physiological data associated with the user from a wearable device, means for correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval, means for selectively modifying the one or more confidence values associated with the one or more candidate taggable events based at least in part on the geographical location data to generate one or more modified confidence values, means for identifying a taggable event of the one or more candidate taggable events within the time interval based at least in part on a modified confidence value associated with the taggable event satisfying a threshold confidence value, and means for causing a GUI of a user device running the application to display an indication of the identified taggable event.

A non-transitory computer-readable medium storing code for identifying taggable events using a wearable device is described. The code may include instructions executable by a processor to receive geographical location data associated with a user throughout a time interval, receive physiological data associated with the user from a wearable device, correlate the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval, selectively modify the one or more confidence values associated with the one or more candidate taggable events based at least in part on the geographical location data to generate one or more modified confidence values, identify a taggable event of the one or more candidate taggable events within the time interval based at least in part on a modified confidence value associated with the taggable event satisfying a threshold confidence value, and cause a GUI of a user device running the application to display an indication of the identified taggable event.

Some examples of the method, apparatuses, and non-transitory computer-readable medium described herein may further include operations, features, means, or instructions for identifying a relationship between the identified taggable event and the physiological data acquired during the time interval, additional physiological data acquired during a different time interval, or both and causing the GUI of the user device to display a message associated with the relationship.

Some examples of the method, apparatuses, and non-transitory computer-readable medium described herein may further include operations, features, means, or instructions for inputting the physiological data and the geographical location data into a machine learning model, wherein correlating the physiological data with the one or more candidate taggable events, selectively modifying the one or more confidence values, identifying the taggable event, or any combination thereof, may be based at least in part on inputting the physiological data and the geographical location data into a machine learning model.

Some examples of the method, apparatuses, and non-transitory computer-readable medium described herein may further include operations, features, means, or instructions for identifying historical taggable event data associated with the user, the historical taggable event data comprising a plurality of historical taggable events identified for the user and historical geographical location data corresponding to the plurality of historical taggable events and identifying that the geographical location data of the user may be associated with the historical geographical location data, wherein selectively modifying the one or more confidence values may be based at least in part on the historical taggable event data and identifying that the geographical location data of the user may be associated with the historical geographical location data.

Some examples of the method, apparatuses, and non-transitory computer-readable medium described herein may further include operations, features, means, or instructions for identifying historical taggable event data associated with the user, the historical taggable event data comprising a plurality of historical taggable events and a time of day in which the plurality of historical taggable events were identified and identifying that the time interval during which the physiological data was acquired may be within the time of day, wherein selectively modifying the one or more confidence values may be based at least in part on the historical taggable event data and identifying that the time interval may be within the time of day.

Some examples of the method, apparatuses, and non-transitory computer-readable medium described herein may further include operations, features, means, or instructions for receiving, via the GUI and based at least in part on displaying the indication of the taggable event, a confirmation of the taggable event, a modification of the taggable event, or both.

In some examples of the method, apparatuses, and non-transitory computer-readable medium described herein, the physiological data comprises at least motion data and the method, apparatuses, and non-transitory computer-readable medium may include further operations, features, means, or instructions for identifying a plurality of motion segments within the time interval based at least in part on the motion data and identifying a gesture the user engaged in based at least in part on matching a motion segment of the plurality of motion segments to a gesture profile of a set of gesture profiles defined within the application, wherein selectively modifying the one or more confidence values, identifying the taggable event, or both, may be based at least in part on identifying the gesture.

Some examples of the method, apparatuses, and non-transitory computer-readable medium described herein may further include operations, features, means, or instructions for identifying a relationship between the identified gesture and the physiological data acquired during the time interval, additional physiological data acquired during a different time interval, or both, wherein correlating the physiological data with one or more candidate taggable events, selectively modifying the one or more confidence values, identifying the taggable event, or any combination thereof, may be based at least in part on identifying the relationship.

In some examples of the method, apparatuses, and non-transitory computer-readable medium described herein, the geographical location data may be associated with a semantic location and the method, apparatuses, and non-transitory computer-readable medium may include further operations, features, means, or instructions for determining that one or more additional users may be located at the semantic location during at least a portion of the time interval, wherein selectively modifying the one or more confidence values, identifying the taggable event, or both, may be based at least in part on determining that one or more additional users may be located at the semantic location during at least a portion of the time interval.

In some examples of the method, apparatuses, and non-transitory computer-readable medium described herein, selectively adjusting a Readiness Score associated with the user, an Activity Score associated with the user, a Sleep Score associated with the user, or any combination thereof, based at least in part on the identified taggable event.

In some examples of the method, apparatuses, and non-transitory computer-readable medium described herein, the geographical location data comprises geographical positioning data acquired via the user device.

In some examples of the method, apparatuses, and non-transitory computer-readable medium described herein, the geographical location data may be received via a calendar application executable by the user device.

In some examples of the method, apparatuses, and non-transitory computer-readable medium described herein, the taggable event comprises a workout, food consumption, caffeine consumption, alcohol consumption, or any combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for identifying taggable events using a wearable device, comprising:
receiving geographical location data associated with a user throughout a time interval;
receiving physiological data associated with the user from a wearable device;
correlating the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval;
selectively modifying the one or more confidence values associated with the one or more candidate taggable events based at least in part on the geographical location data to generate one or more modified confidence values;
identifying a taggable event of the one or more candidate taggable events within the time interval based at least in part on a modified confidence value associated with the taggable event satisfying a threshold confidence value; and
causing a graphical user interface of a user device running the application to display an indication of the identified taggable event.

2. The method of claim 1, further comprising:
identifying a relationship between the identified taggable event and the physiological data acquired during the time interval, additional physiological data acquired during a different time interval, or both; and
causing the graphical user interface of the user device to display a message associated with the relationship.

3. The method of claim 1, further comprising:
inputting the physiological data and the geographical location data into a machine learning model, wherein correlating the physiological data with the one or more candidate taggable events, selectively modifying the one or more confidence values, identifying the taggable event, or any combination thereof, is based at least in part on inputting the physiological data and the geographical location data into a machine learning model.

4. The method of claim 1, further comprising:
identifying historical taggable event data associated with the user, the historical taggable event data comprising a plurality of historical taggable events identified for the user and historical geographical location data corresponding to the plurality of historical taggable events; and
identifying that the geographical location data of the user is associated with the historical geographical location data, wherein selectively modifying the one or more confidence values is based at least in part on the historical taggable event data and identifying that the geographical location data of the user is associated with the historical geographical location data.

5. The method of claim 1, further comprising:
identifying historical taggable event data associated with the user, the historical taggable event data comprising a plurality of historical taggable events and a time of day in which the plurality of historical taggable events were identified; and
identifying that the time interval during which the physiological data was acquired is within the time of day, wherein selectively modifying the one or more confidence values is based at least in part on the historical taggable event data and identifying that the time interval is within the time of day.

6. The method of claim 1, further comprising:
receiving, via the graphical user interface and based at least in part on displaying the indication of the taggable event, a confirmation of the taggable event, a modification of the taggable event, or both.

7. The method of claim 1, wherein the physiological data comprises at least motion data, the method further comprising:
identifying a plurality of motion segments within the time interval based at least in part on the motion data; and
identifying a gesture the user engaged in based at least in part on matching a motion segment of the plurality of motion segments to a gesture profile of a set of gesture profiles defined within the application, wherein selectively modifying the one or more confidence values, identifying the taggable event, or both, is based at least in part on identifying the gesture.

8. The method of claim 7, further comprising:
identifying a relationship between the identified gesture and the physiological data acquired during the time interval, additional physiological data acquired during a different time interval, or both, wherein correlating the physiological data with one or more candidate taggable events, selectively modifying the one or more confidence values, identifying the taggable event, or any combination thereof, is based at least in part on identifying the relationship.

9. The method of claim 1, wherein the geographical location data is associated with a semantic location, the method further comprising:
determining that one or more additional users are located at the semantic location during at least a portion of the time interval, wherein selectively modifying the one or more confidence values, identifying the taggable event, or both, is based at least in part on determining that one or more additional users are located at the semantic location during at least a portion of the time interval.

10. The method of claim 1, further comprising:
selectively adjusting a Readiness Score associated with the user, an Activity Score associated with the user, a Sleep Score associated with the user, or any combination thereof, based at least in part on the identified taggable event.

11. The method of claim 1, wherein the geographical location data comprises geographical positioning data acquired via the user device.

12. The method of claim 1, wherein the geographical location data is received via a calendar application executable by the user device.

13. The method of claim 1, wherein the taggable event comprises a workout, food consumption, caffeine consumption, alcohol consumption, or any combination thereof.

14. An apparatus for identifying taggable events using a wearable device, comprising:
a processor;
memory coupled with the processor; and
instructions stored in the memory and executable by the processor to cause the apparatus to:
receive geographical location data associated with a user throughout a time interval;
receive physiological data associated with the user from a wearable device;
correlate the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval;
selectively modify the one or more confidence values associated with the one or more candidate taggable events based at least in part on the geographical location data to generate one or more modified confidence values;
identify a taggable event of the one or more candidate taggable events within the time interval based at least in part on a modified confidence value associated with the taggable event satisfying a threshold confidence value; and
cause a graphical user interface of a user device running the application to display an indication of the identified taggable event.

15. The apparatus of claim 14, wherein the instructions are further executable by the processor to cause the apparatus to:
identify a relationship between the identified taggable event and the physiological data acquired during the time interval, additional physiological data acquired during a different time interval, or both; and
cause the graphical user interface of the user device to display a message associated with the relationship.

16. The apparatus of claim 14, wherein the instructions are further executable by the processor to cause the apparatus to:
input the physiological data and the geographical location data into a machine learning model, wherein correlating the physiological data with the one or more candidate taggable events, selectively modifying the one or more confidence values, identifying the taggable event, or any combination thereof, is based at least in part on inputting the physiological data and the geographical location data into a machine learning model.

17. The apparatus of claim 14, wherein the instructions are further executable by the processor to cause the apparatus to:
identify historical taggable event data associated with the user, the historical taggable event data comprising a plurality of historical taggable events identified for the user and historical geographical location data corresponding to the plurality of historical taggable events; and
identify that the geographical location data of the user is associated with the historical geographical location data, wherein selectively modifying the one or more confidence values is based at least in part on the historical taggable event data and identifying that the geographical location data of the user is associated with the historical geographical location data.

18. The apparatus of claim 14, wherein the instructions are further executable by the processor to cause the apparatus to:
identify historical taggable event data associated with the user, the historical taggable event data comprising a plurality of historical taggable events and a time of day in which the plurality of historical taggable events were identified; and
identify that the time interval during which the physiological data was acquired is within the time of day, wherein selectively modifying the one or more confidence values is based at least in part on the historical taggable event data and identifying that the time interval is within the time of day.

19. The apparatus of claim 14, wherein the instructions are further executable by the processor to cause the apparatus to:
receive, via the graphical user interface and based at least in part on displaying the indication of the taggable event, a confirmation of the taggable event, a modification of the taggable event, or both.

20. A non-transitory computer-readable medium storing code for identifying taggable events using a wearable device, the code comprising instructions executable by a processor to:
receive geographical location data associated with a user throughout a time interval;
receive physiological data associated with the user from a wearable device;
correlate the physiological data with one or more candidate taggable events of a plurality of candidate taggable events defined within an application associated with the wearable device, the one or more candidate taggable events associated with one or more confidence values that indicate a confidence level that the corresponding candidate taggable events occurred within the time interval;
selectively modify the one or more confidence values associated with the one or more candidate taggable events based at least in part on the geographical location data to generate one or more modified confidence values;
identify a taggable event of the one or more candidate taggable events within the time interval based at least in part on a modified confidence value associated with the taggable event satisfying a threshold confidence value; and
cause a graphical user interface of a user device running the application to display an indication of the identified taggable event.
